(19) United States
(12) Patent Application Publication
(10) Pub. No.: US 2025/0064413 A1
(43) Pub. Date: Feb. 27, 2025

(54) MAMMOGRAM PAD

(71) Applicant: Alberta D. Mitchell, Lilburn, GA (US)

(72) Inventor: Alberta D. Mitchell, Lilburn, GA (US)

(21) Appl. No.: 18/238,435

(22) Filed: Aug. 25, 2023

(51) Int. Cl. A61B 6/04; A61B 6/50

(52) U.S. Cl. CPC A61B 6/0435; A61B 6/502

(57) ABSTRACT

Implementations of a mammogram pad comprising a pad and one or more adhesive tabs forming a perimeter side, a first surface, and a second surface. In some implementations, a method of using the mammogram pad comprises attaching the pad to the breast of a mammogram patient with the adhesive tabs, and further comprises conducting a mammogram on the breast with the pad attached.

### TECHNICAL FIELD

This disclosure relates to implementations of a mammogram pad.

### BACKGROUND

A mammogram is an X-ray picture of the breast. Doctors use a mammogram to look for early signs of breast cancer. Regular mammograms are the best tests doctors have to find breast cancer early, sometimes up to three years before it can be felt. FIG. 1 shows a typical mammogram procedure.

In a typical mammogram procedure, performed with a mammogram machine, a technologist will place the patient's breast on a plastic plate. Another plate will firmly press the patient's breast from above. The plates will flatten the breast, holding it still while an X-ray of the breast is taken.

However, the procedure is usually painful for the patient. For example, the talk around the office (e.g., the doctor's office, clinic, etc.), while women are waiting to get a mammogram, is usually about how painful the mammogram process is in their experience. Furthermore, many women choose to not get mammograms because they are so painful.

### BRIEF DESCRIPTION OF THE DRAWINGS

FIG. 1 illustrates an example existing mammogram procedure.

FIGS. 2A-2I illustrate an implementation of an example mammogram pad according to the present disclosure.

FIGS. 3A-3D illustrate another implementation of an example mammogram pad according to the present disclosure.

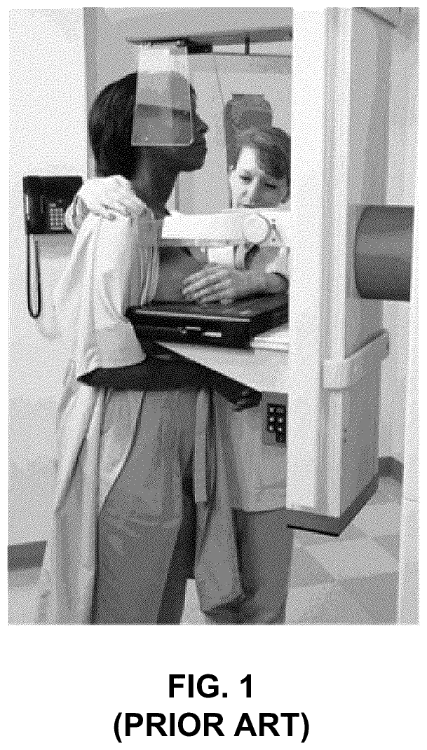

### DETAILED DESCRIPTION

Implementations of a mammogram pad are provided. In some implementations, the mammogram pad comprises a foam pad that sticks on top of a user's breast and one or more adhesive tabs that stick the pad in place on the breast. In some implementations, the mammogram pad comprises a pad, a cover, and one or more adhesive tabs.

In some implementations, the pad, the one or more adhesive tabs, and/or the cover form a perimeter side, a first surface, and a second surface, wherein the first surface is configured to be positioned on a user's breast when the mammogram pad is used.

In some implementations, the mammogram pad is configured to help reduce the pain associated with mammograms.

In some implementations, the mammogram pad is configured to help reduce the pain to a patient caused by a mammogram procedure.

In some implementations, the mammogram pad is configured to help keep a patient comfortable during a mammogram.

In some implementations, the mammogram pad is configured to cover the most sensitive area of a breast during a mammogram.

In some implementations, the mammogram pad is configured to cover a breast during a mammogram without interfering with the mammogram imaging.

In some implementations, the mammogram pad is configured to cushion a breast during a mammogram.

In some implementations, the mammogram pad is a padded, adhesive breast cover made from radiolucent foam and fabric that provides comfort and coverage to a patient's breast during a mammogram.

In some implementations, the mammogram pad is flexible to cover a breast and to allow applying and removing the mammogram pad from the breast.

In some implementations, the mammogram pad is composed of a radiolucent material, such as a radiolucent foam, fabric, and/or adhesive material, such that the material allows the passage of x-rays (X-rays) or other radiation through the material as further described below.

In some implementations, the mammogram pad can be made in a variety of colors and sizes, such as for fit and to enhance a patient's confidence during a mammogram.

In some implementations, a method of using the mammogram pad comprises attaching the pad to the breast of a mammogram patient with the adhesive tabs to provide comfort and coverage to the patient's breast and to help reduce pain during a mammogram. In some implementations, the method further comprises conducting a mammogram on the patient's breast with the mammogram pad attached to the breast.

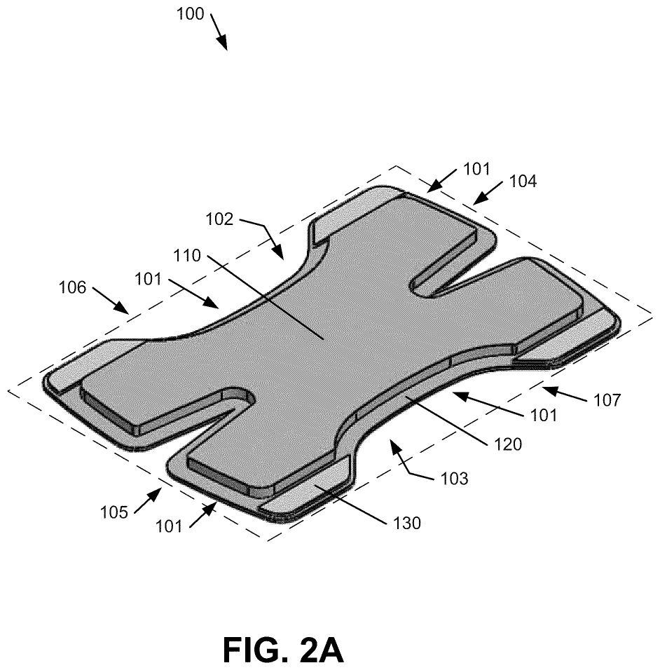

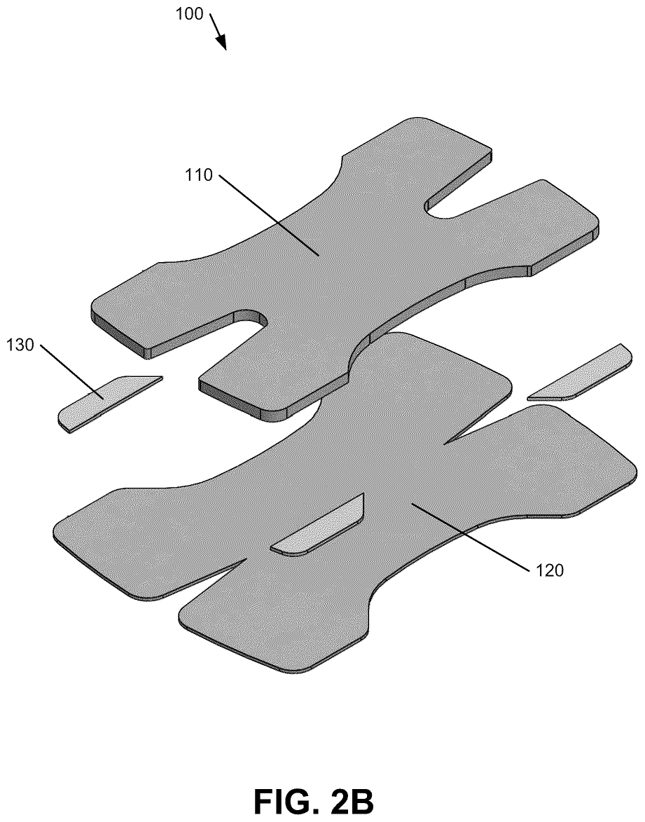

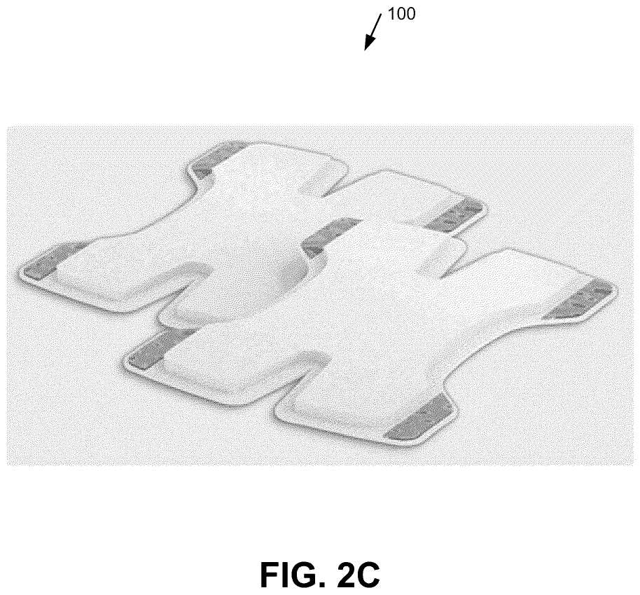

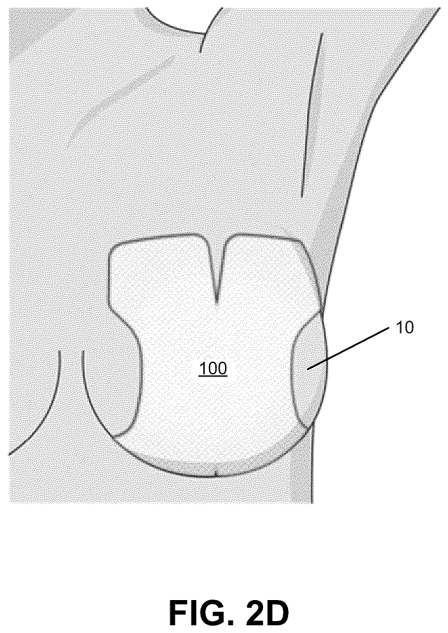

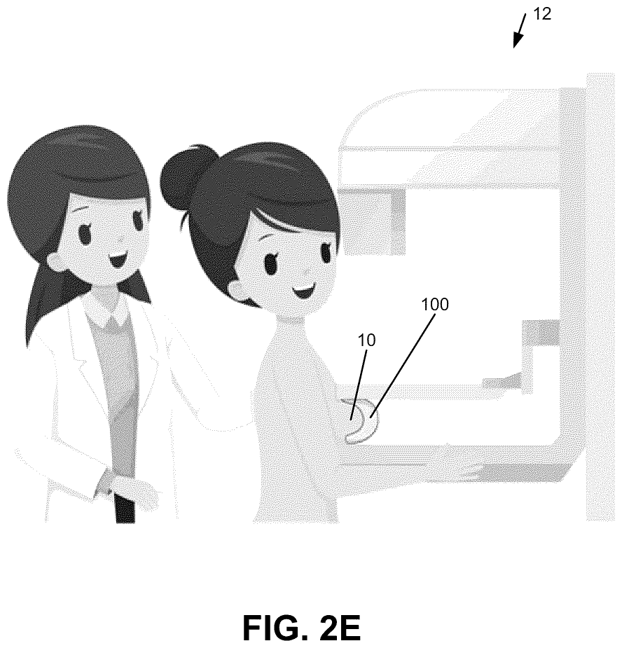

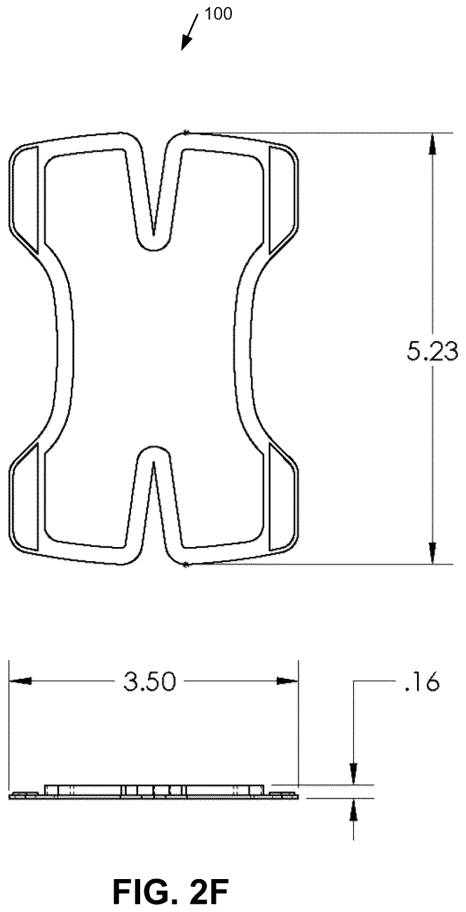

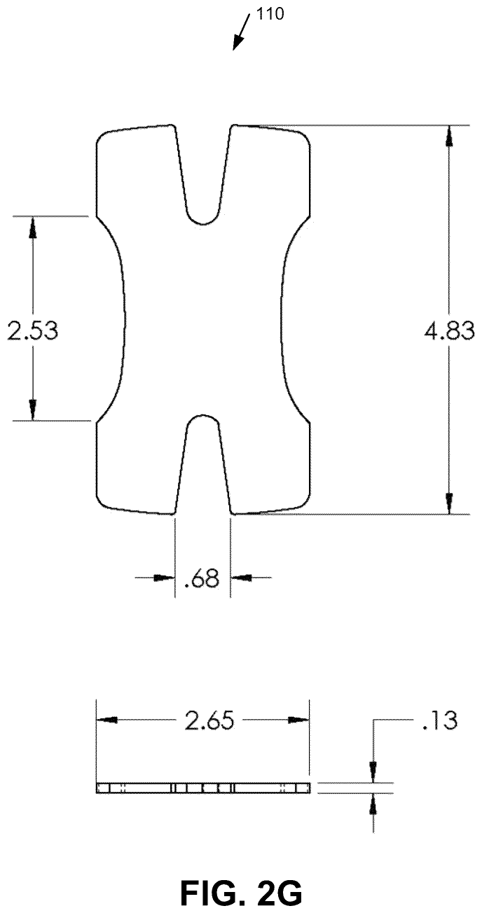

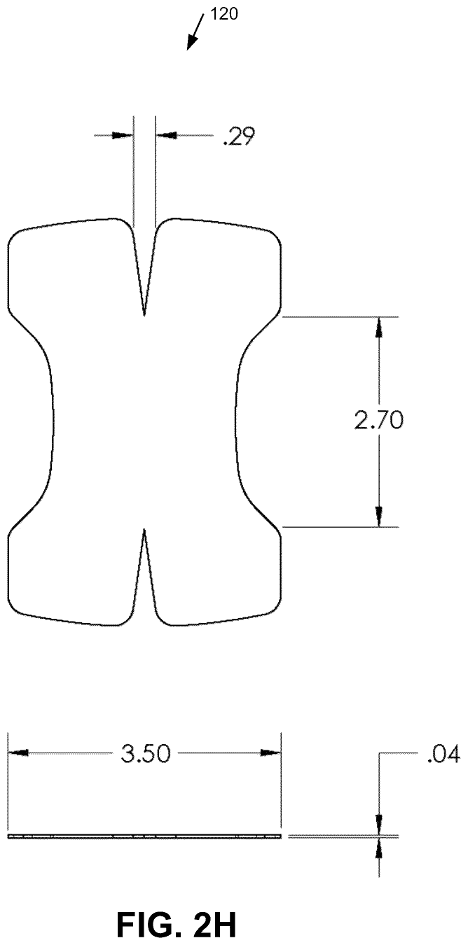

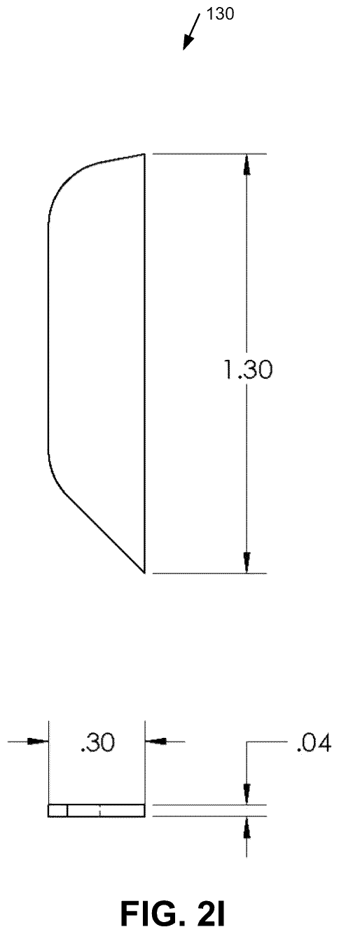

FIGS. 2A-2G illustrate an implementation of an example mammogram pad 100 according to the present disclosure. FIGS. 2A and 2C illustrate perspective views of the mammogram pad 100. FIG. 2B illustrates an exploded perspective view of the mammogram pad 100. FIGS. 2D and 2E illustrate example uses of the mammogram pad 100.

FIGS. 2F-2I illustrate exemplary dimensions of the mammogram pad 100, as further described below. FIG. 2F illustrates exemplary dimensions of a top view and corresponding cross-section view of the mammogram pad 100. FIG. 2G illustrates exemplary dimensions of a top view and corresponding cross-section view of a pad 110 of the mammogram pad 100.

FIG. 2H illustrates exemplary dimensions of a top view and corresponding cross-section view of a cover 120 of the mammogram pad 100. FIG. 2I illustrates exemplary dimensions of a top view and corresponding cross-section view of a adhesive tab 130 of the mammogram pad 100. In some implementations, one or more of the dimensions shown in FIGS. 2F-2I may be less than shown in the figures and/or greater than shown in the figures.

In some implementations, the mammogram pad 100 comprises a foam pad that sticks on top of a breast and one or more (e.g. four) adhesive tabs that stick the pad in place on the breast, for example the pad 110 and the adhesive tabs 130 shown in FIGS. 2A and 2B.

As shown, for example, in FIGS. 2A and 2B, in some implementations, the mammogram pad 100 comprises a pad 110, a cover 120, and one or more (e.g. four) adhesive tabs 130.

In some implementations, the pad 110 may have any suitable configuration (e.g., size and/or shape) such that the mammogram pad 100 can provide comfort and coverage to a patient's breast and help to reduce pain during a mammogram. For example, as shown in FIGS. 2A and 2B, in some implementations, the pad 110 may be generally X-shaped.

As shown in FIGS. 2A and 2B, in some implementations, the pad 110 may be an inner layer or contact layer of the mammogram pad 100. That is, in some implementations, the pad 110 is configured to position on, over, or otherwise in contact with a breast during use of the mammogram pad 100.

In some implementations, the pad 110 is composed of a radiolucent foam material. In some implementations, the pad 110 may be composed of any other suitable material.

As will be understood by one skilled in the art in light of the present disclosure, a radiolucent material, such as the radiolucent foam pad 110, is configured to allow the passage of x-rays (X-rays) or other radiation through the material (i.e., the material is transparent and/or permeable to x-rays and/or other forms of radiation). In this way, in some implementations, the pad 110, as well as other radiolucent components of the mammogram pad 100 (such as the cover 120), is configured not to interfere with x-ray imaging or other processing when used during a mammogram.

In some implementations, the cover 120 may have any suitable configuration (e.g., size and/or shape) such that the mammogram pad 100 can provide comfort and coverage to a patient's breast and help to reduce pain during a mammogram. For example, as shown in FIGS. 2A and 2B, in some implementations, the cover 120 may be generally X-shaped, e.g. similar to the pad 110.

As shown in FIGS. 2A and 2B, in some implementations, the cover 120 may be an outer layer or top layer of the mammogram pad 100. That is, in some implementations, the cover 120 is configured to position such that pad 110 is between the cover 120 and a breast during use of the mammogram pad 100.

As shown in FIGS. 2A and 2B, in some implementations, the cover 120 is attached to the pad 110. In some implementations, the cover 110 comprises a larger, similar shape to the pad 110 such that the perimeter of the sides of the pad 110 position within the perimeter of the sides of the cover 120. In this way, in some implementations, the mammogram pad 100 can be used (e.g., applied and/or removed from a breast) by handling the cover 120 without (or also with) handling the pad 110.

In some implementations, the cover 120 is composed of a radiolucent fabric material (wherein radiolucent material is further described above for the pad 110). In some implementations, the cover 120 may be composed of any other suitable material.

In some implementations, the adhesive tabs (or “tabs”) 130 may have any suitable configuration (e.g., size and/or shape) such that the mammogram pad 100 can be stuck, adhered, or otherwise attached to a patient's breast to provide comfort and coverage and help to reduce pain during a mammogram. For example, as shown in FIGS. 2A and 2B, in some implementations, the adhesive tabs 130 may be generally strip shaped such that the tabs 130 can attach along the sides of the cover 120 and/or pad 110.

As shown in FIGS. 2A and 2B, in some implementations, the tabs 130 are attached to the cover 120, such as on the corners of the cover 120 adjacent to the corners of the pad 110. In some implementations, the tabs 130 may alternately be attached to the pad 100, such as on the corners of the pad 110.

In some implementations, the tabs 130 may be positioned and attached to any other suitable location of the mammogram pad 100.

In some implementations, the tabs 130 are composed of double-sided adhesive tape or similar adhesive material.

In some implementations, the tabs 130 are radiolucent (wherein radiolucent material is further described above for the pad 110).

In some implementations, the tabs 130 may be composed of any other suitable material.

In some implementations, the tabs 130 are configured to stick, adhere, or otherwise attach the mammogram pad 100 to a breast, for example for use of the mammogram pad 100 during a mammogram. In some implementations, the tabs 130 are further configured to remove and/or reattach the mammogram pad 100 to a breast.

In some implementations, the tabs 130 may further comprise a covering, such s a paper or plastic material, to provide a removable protective covering for the exposed adhesive of the tabs 130 that attaches the mammogram pad 100 to the breast.

As shown in FIG. 2A, in some implementations, the mammogram pad 100 (i.e., the combined structure of the pad 110, the cover 120, and the adhesive tabs 130) comprises a perimeter side 101, a first surface 102 (e.g., the pad 110), and a second surface 103 (e.g., the cover 120). In some implementations, the mammogram pad 100 may comprise any other suitable components.

In some implementations, the perimeter side 101 may be any suitable shape. For example, as shown in FIG. 2A, in some implementations, the perimeter side 101 may be generally X-shaped. As indicated in FIG. 2A, in some implementations, the perimeter side 101 may be generally rectangular shaped.

As shown in FIG. 2A, in some implementations, for a generally rectangular shaped perimeter side, a generally opposite first side 104 and second side 105 extend respectively between a generally opposite third side 106 and fourth side 107. Similarly, in some implementations, the generally opposite third side 106 and fourth side 107 extend respectively between the generally opposite first side 104 and second side 105.

In some implementations, the first surface 102 and the second surface 103 may each be any suitable shape. In some implementations, the surfaces 102, 103 are the same or similar shape. For example, as shown in FIG. 2A, in some implementations, the surfaces 102, 103 may be generally X-shaped. As indicated in FIG. 2A, in some implementations, the surfaces 102, 103 may be generally rectangular shaped.

As shown in FIG. 2A, in some implementations, the generally rectangular shaped surfaces 102, 103 extend generally opposite respectively (e.g., lengthwise) between the first side 104 and the second side 105. Similarly, in some implementations, the generally rectangular shaped surfaces 102, 103 extend generally opposite respectively (e.g., widthwise) between the third side 106 and fourth side 107.

As shown in FIG. 2A, in some implementations, the surfaces 102, 103 may extend generally straight or flat from the perimeter side 101. In some implementations, the surfaces 102, 103 may extend in any other suitable configuration between the perimeter side 101.

In some implementations, the first surface 102 is configured to be positioned on or adjacent to a user's breast when the mammogram pad 100 is used. In some implementations, the first surface 102 is configured to thereby face toward the user's skin when the mammogram pad 100 is used.

In some implementations, the first surface 102 may be configured to be positioned in any other suitable way when the mammogram pad 100 is used.

In some implementations, the second surface 103 is configured to be positioned opposite or away from a user's breast when the mammogram pad 100 is used. In some implementations, the second surface 103 is configured to thereby face away from the user's skin when the mammogram pad 100 is used.

In some implementations, the second surface 103 may be configured to be positioned in any other suitable way when the mammogram pad 100 is used.

In some implementations, the mammogram pad 100 may be any suitable shape such that the mammogram pad 100 can be positioned on and attached (e.g., adhered) to a user's breast to provide comfort and coverage to the breast and help to reduce pain during a mammogram (such as shown in FIGS. 2D and 2E). For example, as shown in FIG. 2A, in some implementations, the mammogram pad 100 may be generally X-shaped along the perimeter side 101 and generally flat along the surfaces 102, 103.

In some implementations, the mammogram pad 100 may be any suitable size. For example, in some implementations, the mammogram pad 100 is sized such that the mammogram pad 100 can be positioned on and attached to a user's breast to provide comfort and coverage to the breast and help to reduce pain during a mammogram.

In some implementations, the mammogram pad 100 is configured to help reduce the pain associated with mammograms.

In some implementations, the mammogram pad 100 is configured to help reduce the pain to a patient caused by a mammogram procedure.

In some implementations, the mammogram pad 100 is configured to help keep a patient comfortable during a mammogram.

In some implementations, the mammogram pad 100 is configured to cover the most sensitive area of a breast during a mammogram.

In some implementations, the mammogram pad 100 is configured to cover a breast during a mammogram without interfering with the mammogram imaging.

In some implementations, the mammogram pad 100 is configured to cushion a breast during a mammogram.

In some implementations, the mammogram pad 100 is a padded, adhesive breast cover made from radiolucent foam and fabric that provides comfort and coverage to a patient's breast during a mammogram.

In some implementations, the mammogram pad 100 is flexible to cover a breast and to allow applying and removing the mammogram pad 100 from the breast.

In some implementations, the mammogram pad 100 is composed of a radiolucent material, such as a radiolucent foam, fabric, and/or adhesive material (wherein radiolucent material is further described above for the pad 110).

In some implementations, the mammogram pad 100 can be made in a variety of colors and sizes, such as for fit and to enhance a patient's confidence during a mammogram.

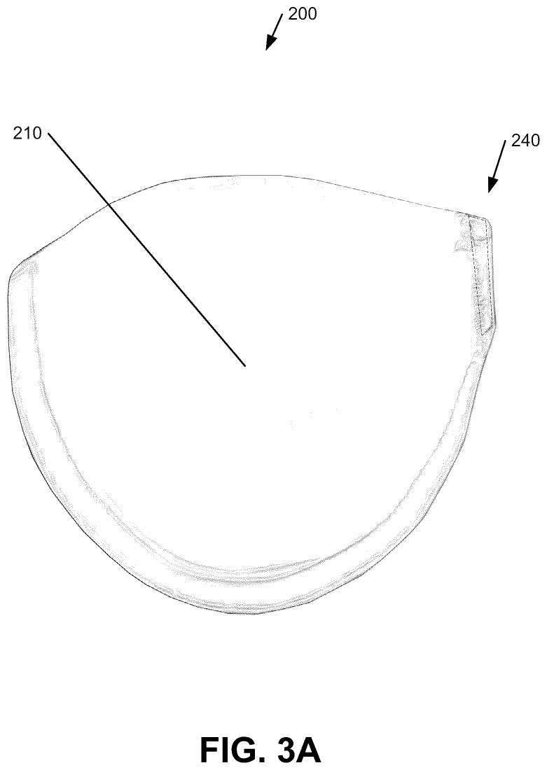

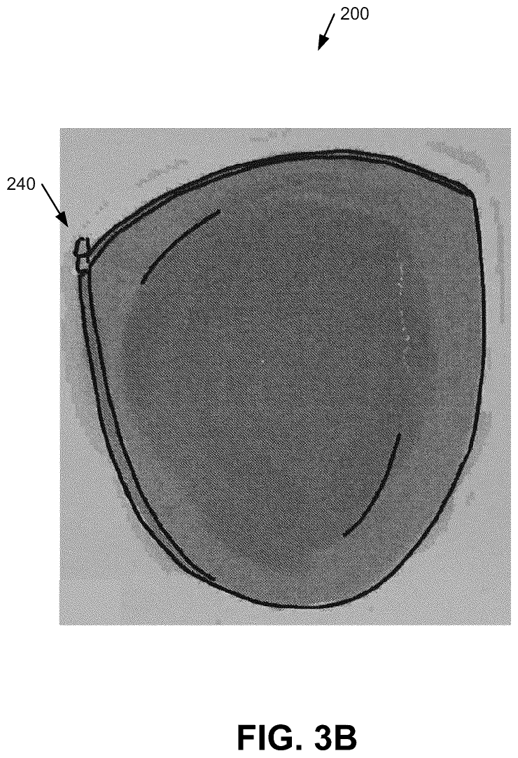

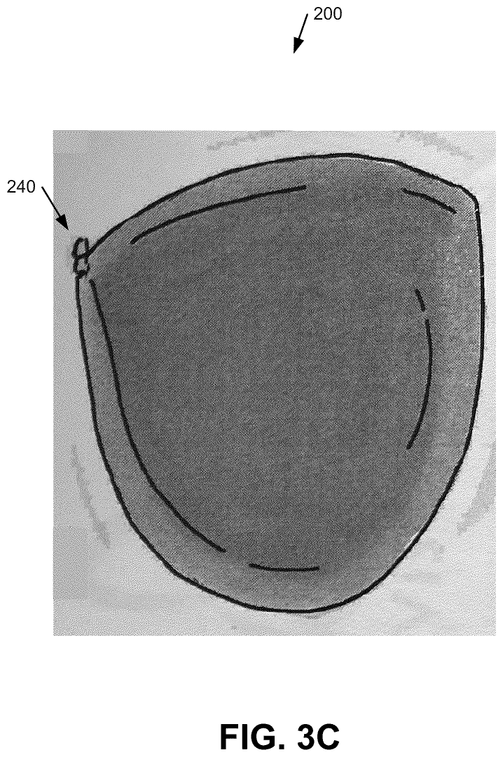

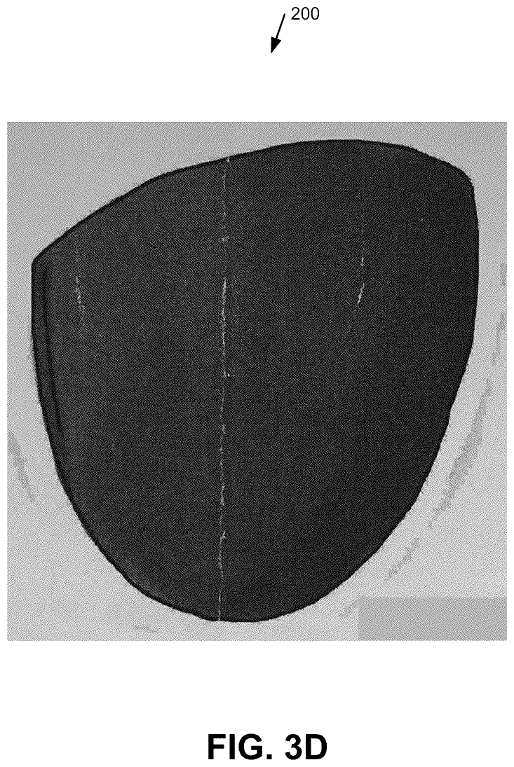

FIGS. 3A-3D illustrate another implementation of an example mammogram pad 200 according to the present disclosure. FIG. 3A illustrates an inner view of the mammogram pad 200. FIG. 3B illustrates an inner view of a deflated mammogram pad 200. FIG. 3C illustrates an inner view of an inflated mammogram pad 200. FIG. 3D illustrates an outer view of the mammogram pad 200.

In some implementations, the mammogram pad 200 is similar to the mammogram pad 100 described herein and shown in FIGS. 2A-2G. For example, in some implementations, the mammogram pad 200 is configured to provide comfort and coverage to a patient's breast and help to reduce pain during a mammogram.

In some implementations, the mammogram pad 200 is composed of a radiolucent material, such as a radiolucent foam, fabric, and/or adhesive material (wherein radiolucent material is further described above for the pad 110). In some implementations, the mammogram pad 200 can be made in a variety of colors and sizes, such as for fit and to enhance a patient's confidence during a mammogram.

As shown, for example, in FIG. 3A, in some implementations, the mammogram pad 200 comprises an inflatable pad 210. In some implementations, the mammogram pad 200 further comprises a valve 240 for inflation of the pad 210.

In some implementations, the mammogram pad 200 may further comprise one or more adhesive tabs that are generally the same or similar to the tabs 130 of the mammogram pad 100.

In some implementations, the pad 210 may have any suitable configuration (e.g., size and/or shape) such that the mammogram pad 200 can provide comfort and coverage to a patient's breast and help to reduce pain during a mammogram. For example, as shown in FIG. 3A, in some implementations, the pad 210 may be generally breast-cup shaped.

As shown in FIGs. FIG. 3A, in some implementations, the pad 210 is configured to position on, over, or otherwise in contact with a breast during use of the mammogram pad 200.

In some implementations, the pad 210 is composed of radiolucent material (wherein radiolucent material is further described above for the pad 110), including material configured to be inflated for use of the mammogram pad 200. In some implementations, the pad 210 may be composed of any other suitable material.

In some implementations, the valve 240 may be any other suitable valve that allows the inflation (and, in some implementations, the deflation) of the mammogram pad 200. In some implementations, the valve 240 may have any other suitable configuration (e.g., size and/or shape).

In some implementations, the valve 240 may be composed of radiolucent material (wherein radiolucent material is further described above for the pad 110). In some implementations, the valve 240 may be composed of any other suitable material.

In some implementations, the valve 240 may be positioned on the mammogram pad 200 such that it does not interfere with x-ray imaging or other processing when the mammogram pad 200 is used during a mammogram.

In some implementations, the mammogram pad 200 is configured to inflate to gently secure in place around a breast. In this way, in some implementations, the mammogram pad 200 provides a layer of cushioning between the breast and a mammogram device to provide a patient with comfort and coverage and to help reduce pain during a mammogram.

In some implementations, the mammogram pad 200 is further configured to inflate to adjust the size of the pad 210 to better fit a breast for use of the mammogram pad 200.

In some implementations, the mammogram pad 200 is further configured to deflate, for example from the valve 240.

In some implementations, the mammogram pad 200 is configured to inflate, such as shown in FIG. 3C, for use of the mammogram pad 200 and to deflate, such as shown in FIG. 3B, for storage of the mammogram pad 200.

In some implementations, the mammogram pad 200 may be further configured to attach to a breast by one or more adhesive tabs that are generally the same or similar to the tabs 130 of the mammogram pad 100.

In some implementations, another implementation of a mammogram pad according to the present disclosure may comprise the pad 110 of the mammogram pad 100 and/or the pad 210 of the mammogram pad 200, as described herein, retrofitted to a mammogram machine such that the mammogram pad helps to reduce pain during a mammogram with the machine. In this way, the mammogram pad can provide this and other features described herein of the mammogram pad 100 and/or the mammogram pad 200 during use of the mammogram device.

In some implementations, the mammogram pad 100, 200 comprises any suitable dimensions, such as the example dimensions of the mammogram pad 100 shown in FIGS. 2F-2I. In some implementations, one or more of the dimensions shown in FIGS. 2F-2I may be less than shown in the figures and/or greater than shown in the figures.

In some implementations, the mammogram pad 100, 200 is composed of any suitable materials. For example, as described above, in some implementations, the mammogram pad 100, 200 is composed of a radiolucent material, such as a radiolucent foam, fabric, and/or adhesive material (wherein radiolucent material is further described above for the pad 110).

In some implementations, the mammogram pad 100, 200 can have any suitable appearance, such as shown in the figures.

FIGS. 2D and 2E illustrate example uses of the mammogram pad 100 according to the present disclosure.

In some implementations, an example method of using the mammogram pad 100, with respect to the above-described figures, comprises attaching the mammogram pad 100 to the breast 10 of a mammogram patient, such as shown in FIG. 2D. For example, in some implementations, the pad 110 of the mammogram pad 100 is attached to the breast by the adhesive tabs 130. In some implementations, a protective covering may be removed (e.g., peeled off) from the tabs 130 to expose the adhesive for use.

In some implementations, the mammogram pad 100 is attached to the breast by handling the cover 120 without (or also with) handling the pad 110.

In some implementations, the attached mammogram pad 100 is used to provide comfort and coverage to a patient's breast and to help reduce pain during a mammogram.

In some implementations, the method further comprises conducting a mammogram on the patient's breast 10 with a mammogram machine 12 while the mammogram pad 100 is attached to the breast 10, such as shown in FIG. 2E.

In some implementations, the method may further comprise removing the mammogram pad 100 from the breast. For example, in some implementations, the mammogram pad 100 is detached from the breast by unadhereing the tabs 130.

In some implementations, an example method of using the mammogram pad 200, with respect to the above-described figures, comprises attaching the mammogram pad 200 to the breast of a mammogram patient. For example, in some implementations, the mammogram pad 200 is attached to the breast by inflating the pad 210 to gently secure the pd 200 in place around the breast.

In some implementations, the pad 210 is inflated using the valve 240.

In some implementations, the attached mammogram pad 200 is used to provide comfort and coverage to a patient's breast and to help reduce pain during a mammogram.

In some implementations, the method further comprises conducting a mammogram on the patient's breast with a mammogram machine while the mammogram pad 200 is attached to the breast.

In some implementations, the method may further comprise removing the mammogram pad 200 from the breast. For example, in some implementations, the mammogram pad 200 is deflated to unsecure the pad 210 from the breast.

In some implementations, securing and unsecuring the mammogram pad 200 from the breast may further comprising adhesive tabs used similar to as described above for the mammogram pad 100.

In some implementations, the method may further comprise storing the deflated mammogram pad 200.

The figures, including photographs and drawings, comprised herewith may represent one or more implementations of the mammogram pad.

Details shown in the figures, such as dimensions, descriptions, etc., are exemplary, and there may be implementations of other suitable details according to the present disclosure.

Reference throughout this specification to “an embodiment” or “implementation” or words of similar import means that a particular described feature, structure, or characteristic is comprised in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations may be depicted in the drawings in a particular order, this should not be understood as requiring that such operations be performed in the particular order shown or in sequential order, or that all illustrated operations be performed, to achieve desirable results.

1. An article of manufacture comprising:
a piece of material having a perimeter side, a first surface, and a second surface, the piece of material forming a mammogram pad configured to cushion a user's breast to help reduce pain to the breast caused by a mammogram wherein:
the piece of material is sized and shaped to position on a user's breast;
the perimeter side defines the perimeter of the piece of material extending between the first and second surfaces;
the first and second surfaces extend opposite to each other between the perimeter side; and
the piece of material comprises a radiolucent foam pad configured not to interfere with the x-ray imaging of a mammogram.

2. The article of manufacture of claim 1 wherein the piece of material further comprises an adhesive such that the piece of material is adherable and removable from adherence to a user's skin when the piece of material is positioned on the user's breast.

3. The article of manufacture of claim 1 wherein
the first surface comprises the radiolucent foam pad configured not to interfere with the x-ray imaging of a mammogram;
and the second surface comprises a radiolucent piece of fabric configured not to interfere with the x-ray imaging of a mammogram.

4. The article of manufacture of claim 1 wherein the perimeter side is X-shaped.

5. The article of manufacture of claim 1 wherein the piece of material has a length of about 5.23 inches, a width of about 3.5 inches, and a thickness between the first surface and the second surface of about 0.16 inches.

6. A method of using the article of manufacture of claim 1 comprising positioning the piece of material on a user's breast such that the first surface faces toward the user's skin to cushion the user's breast to help reduce pain to the breast caused by a mammogram.

7. The method of claim 6 further comprising conducting a mammogram on the user's breast with a mammogram machine while the piece of material is positioned on the user's breast.

8. The method of claim 6 further comprising removing the piece of material from the positioning on the user's breast.

9. A method of using the article of manufacture of claim 2 comprising positioning and adhering the piece of material on a user's breast such that the first surface faces toward the user's skin to cushion the user's breast to help reduce pain to the breast caused by a mammogram.

10. The method of claim 9 further comprising conducting a mammogram on the user's breast with a mammogram machine while the piece of material is positioned and adhered on the user's breast.

11. The method of claim 9 further comprising removing the piece of material from the user's breast.
